(19) United States
(12) Patent Application Publication
(10) Pub. No.: US 2008/0119664 A1
(43) Pub. Date: May 22, 2008

(54) OPTIMIZED BIODIESEL REACTION KINETICS SYSTEM

(76) Inventors: Jacques Sinoncelli, San Anselmo, CA (US); David R. Wallace, San Francisco, CA (US)

Correspondence Address: WINSTON & STRAWN LLP;PATENT DEPARTMENT, 1700 K STREET, N.W., WASHINGTON, DC, 20006, US

(21) Appl. No.: 11/928,243

(22) Filed: Oct. 30, 2007

Related U.S. Application Data
(60) Provisional application No. 60/855,204, filed on Oct. 30, 2006.
(60) Provisional application No. 60/874,595, filed on Dec. 13, 2006.
(60) Provisional application No. 60/902,179, filed on Feb. 20, 2007.

(51) Int. Cl. C07C 51/43

(52) U.S. Cl. 554/176

(57) ABSTRACT

A method for making alkyl esters, such as methyl ester, for use as a biodiesel fuel from various oil sources. A steady state reaction with a single phase solution created from multi phase constituents can be achieved without the use of co-solvents that cannot or should not be present in the final fuel and must therefore be removed.

### CROSS-REFERENCE TO RELATED APPLICATIONS

This application claims the benefit of U.S. Provisional Patent Application No. 60/855,204, filed Oct. 30, 2006 entitled “DRY-PROCESS BIODIESEL PRODUCTION” to Michael B. Brown et al., U.S. Provisional Patent Application No. 60/874,595, filed Dec. 13, 2006 entitled “ION-RESIN BIODIESEL PRODUCTION” to Michael B. Brown et al. and U.S. Provisional Patent Application No. 60/902,179, filed Feb. 20, 2007 entitled “STEADY-STATE BIODIESEL SYSTEM” to Michael B. Brown et al., which are hereby incorporated in the entirety by this reference.

### FIELD OF THE INVENTION

This invention is concerned with methods for producing biodiesel fuels from various oils and fats.

### BACKGROUND OF THE INVENTION

Natural fats and oils consist of 95 to 98 per-cent triglycerides, which can be transformed by transesterification into biodiesel fuel. Alcohol and triglycerides react in the presence of a strong base, acting as a catalyst, producing alkyl ester, i.e. the biodiesel, and glycerin. The most common alcohol used is methanol in which case methyl ester is produced. The strong base catalyst most commonly used is sodium hydroxide, although there are now available various heterogeneous, or hard, catalysts.

Many of the current methods for producing biodiesel date from the 1940's. Most are batch methods that have serious inefficiencies. There are some methods that use a continuous flow approach, but they are still based mostly on the inefficient batch process.

### SUMMARY OF THE INVENTION

A biodiesel reaction has a slow initial stage which is mass transfer limited. Initially, the reactants methanol and triglyceride are in two separate liquid phases that are immiscible. However, as the reaction progresses, methyl ester is formed in the reaction causing a transition to a single-phase kinetically controlled system, whereupon the reaction rate accelerates significantly.

Approaches to solve this problem of the initial slow reaction have included turbulent or high-shear mixing as well as the addition of a co-solvent, such as tetrahydrofuran. High-shear mixing is still relatively slow and co-solvents must be removed at a later stage. Typically co-solvents also require hazardous materials handling procedures.

Instead, embodiments of this invention bypass this initial slow mixing stage by creating a closed steady-state subsystem containing the combination of components found in the later fast single-stage reaction. Since in the later stages the methanol and triglyceride are partially reacted to form methyl ester, the system simulates that stage of the reaction by adding methyl ester to the input components. With the correct proportion of methanol, triglyceride and methyl ester, the normally immiscible multi phase constituents will go into a single phase solution. When the reactants are in a single phase solution they can react immediately at a molecular level, providing a much faster, more complete reaction.

In the case of a transesterification reaction, the primary reaction results are methyl ester and glycerin. The methyl ester goes into solution as it is produced, while the glycerin instead forms an emulsion within the existing solution.

The input and output flows for the subsystem are managed in order to preserve a fixed proportion of the constituents within the closed-cycle subsystem. In particular, note that glycerin is removed from the system as one of the measures to keep the proportion of constituents fixed. The relative proportion of constituents in the steady-state subsystem, as well as its temperature and pressure, can then be optimized relative to reaction speed, reaction completeness or other characteristics. In other words, this approach allows the optimization of the biodiesel reaction dynamics.

In certain embodiments the system at startup may be primed with methyl ester input into the mixer from a separate source until sufficient methyl ester is produced by the reactor to attain the desired level of methyl ester by recirculation. Alternatively, the methyl ester output from the subsystem can be constrained until the methyl ester input into the mixer builds up to the right level.

The closed subsystem includes a catalytic reactor to accelerate the reaction, a glycerin extraction stage and an outlet from the closed system for downstream subsequent processing. The closed cycle reactor includes glycerin removal to bias the equilibrium of the reaction towards methyl ester production.

The reactor can include various types of solid catalyst, including base resin beads, such as Amberlyst A26(OH). The reactor can also include microwave irradiation of the contained reactants. The reactor is also managed to control temperature, pressure and flow.

Heterogeneous catalysts have been a problem in the past because of their slow reaction times. This is primarily caused by the immiscible reactants that flow past the solid catalyst in separate chunks or bubbles that can only react on the surfaces or interfaces between those reactants. The current system solves this problem by delivering the reactants to the catalyst surface area already mixed at the molecular level.

A liquid or homogeneous catalyst, such as sodium methylate, could be used in this system as well. The disadvantage of sodium hydroxide based homogeneous catalysts is the presence of free sodium ions which react with FFA in the feedstock to create soaps. This is a serious problem for the traditional downstream water wash approaches because the soaps typically foam up in the washing process. However, the current system avoids this problem by having a completely water-free process.

First, the system uses a thorough drying process for the feedstock. Then for the catalyst, the system uses water-free sodium methylate. In contrast, the traditional approach is to mix methanol and sodium hydroxide which reacts to form sodium methylate along with water. This water can then side track the desired transesterification reaction resulting in FFA instead of methyl ester, causing even more soap creation.

Further, when the system uses the optional acid reactor to deal with high-FFA feedstock, it uses distillation post processing to eliminate the water produced by the esterification that transforms the FFA into methyl ester.

Additionally, the system uses a post processing “dry wash” step that converts soap back to FFA. Soap is not allowed in stringent biodiesel fuel standards, whereas a certain amount of FFA is permissible. In the preferred embodiments the “Dry Wash” consists of passing the unprocessed biodiesel through a column of ion-resin beads, for instance, Amberlite™ BD10DRY™ from Rohm and Haas. This Dry Wash step also adsorbs any remaining sodium hydroxide catalyst as well as filtering out remaining glycerin. The glycerin filtration is mechanical instead of being ion-based and results from the small size of the beads and their configuration in a bed.

Further, when high-FFA feedstocks are used, certain embodiments use, as a pre-processing step, a type of heterogeneous acid catalyst technology that avoids the use of, and complications related to, the sulfuric acid required by the traditional homogeneous approach. However, alternative embodiments may not employ such a heterogeneous acid catalyst for preprocessing high-FFA feedstocks, and other techniques may be employed.

In some embodiments, a strong acid heterogeneous catalyst contains acid molecular components that are exposed to provide the catalytic function provided by the free sulfuric acid of prior techniques.

Consequently, the FFA mixed with methanol can react as it passes through a catalyst bed without the need for tanks and components resistant to acid and without the need to remove the acid afterwards. Such catalysts can include sulfated zirconia or ion-resin beads. During tests of sulfated zirconia for pre-processing a feedstock containing 14% FFA, the solution-based approach of mixing feedstock, methanol and methyl ester resulted in a reaction that rapidly reduced the FFA level to 5.4%.

Please note that although methanol use is described in this system, other alcohols, such as ethanol, could be used instead.

### BRIEF DESCRIPTION OF THE DRAWINGS

FIG. 1 is a schematic diagram of an embodiment of a biodiesel reactor system.

FIG. 2 shows the closed cycle system of the base reactor 114 for transesterification when a heterogeneous catalyst is used.

FIG. 3 shows a closed cycle system of the acid reactor 109 for esterification of FFA.

FIG. 4 shows a closed cycle system of the base reactor for transesterification when a homogeneous catalyst is used.

FIG. 5 shows the heterogeneous version of the finish reactor.

FIG. 6 shows the homogeneous version of the finish reactor.

### DETAILED DESCRIPTION OF THE PREFERRED EMBODIMENTS

Prior approaches of producing biodiesel fuel allow water to be introduced into the process and also follow the process with a water wash to remove excess alcohol and catalyst. The presence of water during reaction can produce soaps which seriously reduces yields and requires post processing. Further, biodiesel with too much water content deteriorates quickly and is hygroscopic making water removal difficult once it is present.

Sodium hydroxide in prior processes reacts with the triglycerides, not only allowing the formation of soaps, but also consuming sodium hydroxide as a constituent of an undesirable reaction instead of allowing the sodium hydroxide to act as a catalyst for esterification or transesterification. Further, the standard practice of mixing sodium hydroxide with methanol is not only dangerous, but the resulting reaction produces not only methylate, which is desired, but also water, which is undesirable as previously mentioned.

Another complication that may be present in biodiesel processing is the presence in certain feedstocks of too much free fatty acid (“FFA”). This is not a problem with refined vegetable oils, such as soybean oil or rapeseed oil, but can be a problem for cheaper waste-derived feedstock. Fatty acids can be bound or attached to other molecules, such as in triglycerides or phospholipids. When they are not attached to other molecules, they are known as “free” fatty acids.

When feedstock containing FFA is processed with transesterification using sodium hydroxide, the FFA immediately combines with the sodium ions to make soap.

Thus feedstocks with more than 5% free fatty acids, which include animal fats and recycled greases, are pretreated in an acid esterification process. In this step, the feedstock is reacted with methanol in the presence of a sulfuric acid catalyst, converting the free fatty acids into biodiesel methyl ester, and water.

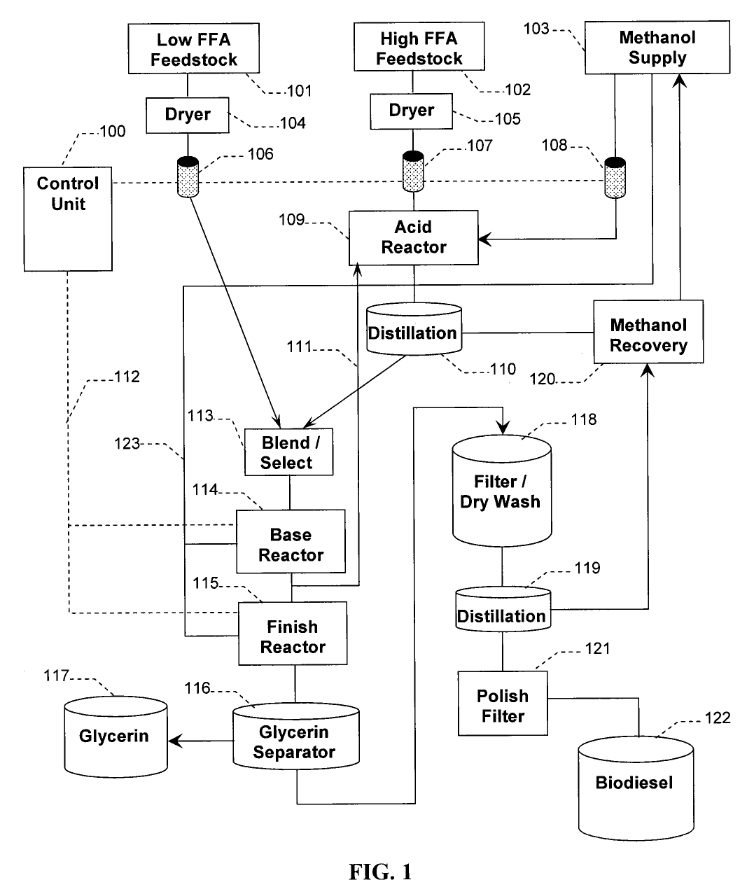

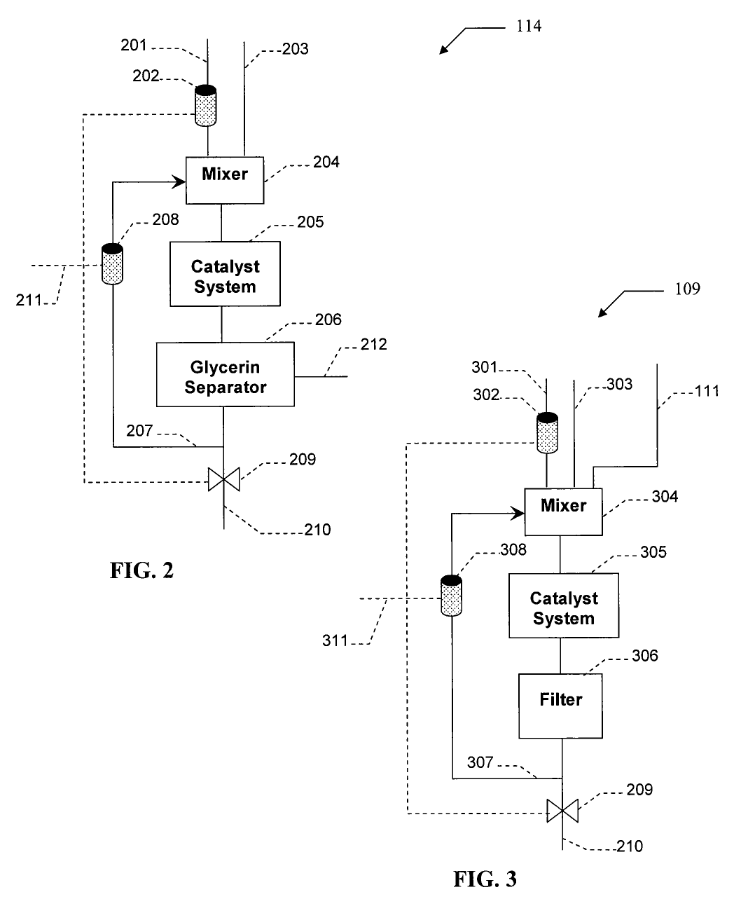

FIG. 1 is a schematic diagram of an embodiment of a biodiesel reactor system. FIG. 2 shows the closed cycle system of the base reactor 114 for transesterification when a heterogeneous catalyst is used.

Element 100 is the computer control unit that manages the various metering pumps and valves throughout the system. The metering pumps provide accurate flow and pressure to ensure both the accurate proportions of ingredients to be mixed as well as the proper operation of glycerin separators, such as element 206 or optionally element 116, which provide precision separation of reaction components. The dotted lines show the control connections to the metering pumps, such as elements 106, 107, 108, 202 and 302.

Element 101 is the tank for feedstock which contains of oil such as refined soybean oil or other oil with a free fatty acid content of less than about five per-cent. These oils can proceed to the transesterification process without separate FFA processing.

Element 102 is the optional tank for oil or fat with free fatty acid, FFA, content about five per-cent or greater. Typically waste oils and fats have a high free fatty acid content and also need to be filtered before processing. High FFA oils and fat need an esterification pre-processing step to lower the FFA level. To avoid the various problems associated with the use of sulfuric acid, the current invention uses a solid heterogeneous catalyst instead. The current preferred embodiment of the invention uses sulfated zirconia. However, other strong acid catalysts, such as Amberlyst™ 70 from Rohm and Haas may be utilized.

The high FFA feedstock in 102 is first dried by the dryer at 105 and then with the methanol from the supply at 103 are sent to the acid reactor at 109. The dryer recirculates and heats the feedstock under a vacuum to remove any residual moisture.

The two metering pumps 107 and 108 supply the feedstock and methanol to the reactor in the correct proportions and the supply line 111 provides methyl ester to that reactor. This connection may need to be primed to get the process started. The methyl ester is of a fatty acid and may be referred to as a fatty acid methyl ester. The stoichiometric ratio of oil to methanol in this reaction is about 9.2 by weight depending on the oil. However, to push this reaction in the desired direction, up to double the amount of methanol is usually employed, which would be an oil to methanol ratio of up to 4.6 by weight. The acid reactor 109 will be described in further detail with regard to FIG. 3.

After the acid reactor there is distillation at distiller 110 to remove the water resulting from the esterification reaction in reactor 109. This distillation will require removing both the water and methanol from the output and separating them as part of the process.

The low FFA feedstock is dried at dryer 104. The metering pump 106 controls the proportion of the low FFA feedstock that is then optionally mixed with the preprocessed constituents coming from the acid reactor 109 via distiller 110. At blender 113 the two different feed inputs can be either blended together or chosen selectively, if only one kind of feedstock is being used.

Output from the acid reactor will already contain a certain proportion of methyl ester both from the catalyst reaction itself as well as the methyl ester added to create a substantially single phase solution. The control unit will make appropriate adjustments to downstream additions of methyl ester to compensate for this.

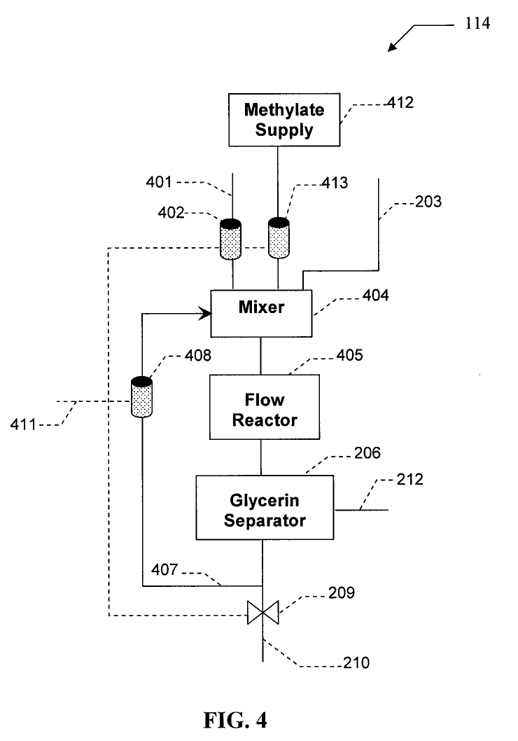

The blended or selected constituents then go to the base reactor 114. Methanol from supply 103 is carried to the base reactor via the supply line 123. At base reactor 114 transesterification is performed using a base catalyst. Details for a reactor using a heterogeneous catalyst are in FIG. 2 and for a homogeneous one in FIG. 4.

The output from the base reactor is primarily biodiesel methyl ester, but it may not be completely reacted. In some embodiments it is followed by a finish reactor 115 designed to complete the transesterification reaction. This reactor is supplied with additional methanol via supply line 123.

This is followed by glycerin separation at separator 116. Separated glycerin is sent to glycerin storage tank 117. Glycerin removal can be accomplished with a centrifuge, settling tanks or other means. If a centrifuge is used, it is tuned to cut a small amount of biodiesel along with the glycerin, leaving a cleaner biodiesel. This glycerin byproduct carries away trace amounts of biodiesel and methanol.

After the glycerin removal at separator 116, the biodiesel reaction result then goes on to cleanup phases. If the base reactor 114 has used a heterogeneous catalyst, the element at 118 may be a filter, as a dry wash is not needed, although it may be performed for surety. A heterogeneous catalyst does not produce soaps.

If the catalyst used in reactor 114 is homogeneous, then a “dry wash” is used to remove residual soaps. Prior processes use a water wash at this point which requires a subsequent water removal with the risk of biodiesel contamination. In embodiments of the invention the use of active ion resin material eliminates the need for a water wash and the need for subsequent water removal.

The dry wash process uses ion-resin beads. The preferred embodiment of the invention uses Amberlite BD10DRY™ ion resin beads provided by Rohm and Haas. The biodiesel enters the top of a tower filled with ion resin exchange beads and flows down through the cylinder where the active material of the beads reacts with any remaining soaps, turning them into FFA, as well as adsorbing any remaining catalyst. The tower of dry wash ion-resin beads also acts as a mechanical filter to trap impurities as well as possible remaining glycerin. The result is a biodiesel that is pure and dry.

The ion resin beads typically need replacing at the rate of about one metric ton for each 250,000 gallons of biodiesel processed. Once their lifespan is over, the beads are neutral and non-toxic so may be disposed of easily.

Since the dry wash or filter does not remove methanol, it is followed by a distillation process at distiller 119 to remove any remaining methanol. The methanol separation is done by heating the reacted constituents to over 100 degrees centigrade while under a vacuum. The intermediate product filters down over heated dispersion plates. The separated biodiesel flows out of the lower portion of the unit while the vaporized methanol is drawn off the top and sent to the methanol recovery unit 120. If there are any trace amounts of water left after the dry wash, they are separated out with the methanol and sent to the methanol recovery unit.

Methanol recovery takes the vapor from distiller 119 and separates any water vapor from methanol vapor and then condenses each. Any trace amount of water extracted is pure and may be disposed of in many ways. The extracted methanol is sent back to the methanol supply 103.

After distillation at distiller 119, the biodiesel product proceeds to the final polishing filter 121 which removes any extremely fine particulates that may have made it through the process to this point. The final biodiesel product is now ready and is sent to a storage tank at 122.

FIG. 2 illustrates a heterogeneous version of the base (transesterification) reactor 114 from FIG. 1. The processed feedstock entering the base reactor via 203 goes to the mixing unit 204. The mixing unit combines methanol from line 201, metered in proper proportion by the metering pump 202. Mixing unit 204 also combines the methyl ester coming from the recirculating pump 208 via supply line 207.

Mixing unit 204 may be static or dynamic and of any number of geometries, but is preferably a commercially available static mixer. A steady state of constituents with sufficient methyl ester content to create and preserve a solution is maintained by controlling by how much methyl ester is recirculated by pump 208 via line 207, in proportion to the inputs 201 and 203 with control unit 100 (connected via the control line 211). At a minimum of 60° C. and at least atmospheric pressure, about 15% methyl ester has been shown to be sufficient to create and preserve the substantially single phase solution.

The mixed constituent solution then passes from the mixer to the heterogeneous catalyst (reactor) system, 205. This catalyst system can be a column or bed which provides a strong-base ionic environment through which the feedstock and methanol pass. The current preferred embodiment of the invention uses, by way of example, Amberlyst A26 OH resin ion beads from Rohm and Haas. However, other strong base catalysts may be employed.

Next glycerin is separated at separator 206. This may be done by centrifuge, settling tanks or other means. The separated glycerin goes out on line 212 to glycerin storage at 117.

Since transesterification is an equilibrium reaction, removal of glycerin at this stage forces the following reaction to be a more complete transesterification. Back pressure valve 209 is used to control the flow rate out of the reactor in combination with the upstream metering pumps, valve settings and control system. The output proceeds via line 210 which connects to lines 111 and 115.

Please note that the system presented here is the preferred embodiment and that the invention is intended to cover other arrangements of components. For instance, the input to the recirculation loop 207 could be placed at a different stage of the process, such as later, as long as it returns sufficient methyl ester for the mixer to create a substantially single phase solution.

FIG. 3 illustrates acid (esterification) reactor 109 from FIG. 1. The dried feedstock, entering the acid reactor via 303, goes to the mixing unit 304. The mixing unit also combines methanol from line 301, connected to line 123, metered in proper proportion by the metering pump 302. The mixing unit also mixes in methyl ester, from supply line 111, sufficient to create a single phase solution of all constituents, using the same proportion as used by mixing unit 204.

The constituent solution then passes from the mixer to the heterogeneous catalyst (reactor) system, 305, which does esterification of FFA.

This catalyst system can be a column or bed which provides a strong-acid ionic environment through which the feedstock and methanol pass. The current preferred embodiment of the invention uses sulfonated zirconia, although other strong acid catalysts can be used, such as Amberlyst 70 resin ion beads from Rohm and Haas.

Again, the fact that the constituents are in substantially a single phase rather than an immiscible or multi-phase status significantly increases the reaction speed and throughput of the system.

In the traditional biodiesel process, the added catalyst, H₂SO₄, is removed in the subsequent transesterification stage. The sulfate ion in the sulfuric acid combines with the sodium ion in the lye during the biodiesel transesterification reaction to form sodium sulfate, which is a water-soluble salt and is removed in a following water wash. Again, embodiments of the present invention avoid water and its associated complications.

The output from the catalyst goes through the filter 306 and is optionally recirculated via line 307 under control of the recirculation pump 308. This recirculation allows more contact of constituents with the catalyst surface, enabling a more complete reaction. The unit 309 is a back pressure valve which helps control the flow rate out of reactor via line 310.

Please see FIG. 4 which describes the homogeneous version of the base reactor 114 from FIG. 1. Please refer to the prior description of components described with regard to prior Figures.

The processed feedstock entering the base reactor via 403 goes to the mixing unit 404. There methylate catalyst is mixed-in in a proportion controlled by the metering pump 413 from the supply 412. The methylate in 412 is premixed and dry to avoid introducing unwanted water.

The mixing unit 404 also combines methanol from line 401, connected to line 123, metered in proper proportion by the metering pump 402. The mixing unit also mixes in methyl ester sufficient to create a substantially single phase solution of all constituents, using the same proportion and conditions as used by the mixing unit 204. The methyl ester comes from the supply line 407 driven by the recirculating pump 408. The control unit 100 manages the metering and recirculating pumps via the control line 411 connected to 112.

The mixed constituents in solution then pass to the flow reactor, 405, which provides static mixing in a particular temperature and pressure environment. After glycerin separation, the recirculation line 407 goes to the recirculation pump 408. The separated glycerin goes out on line 412 to glycerin storage at 117. Back pressure valve 409 is similar to back pressure valve 209. The output proceeds via line 410.

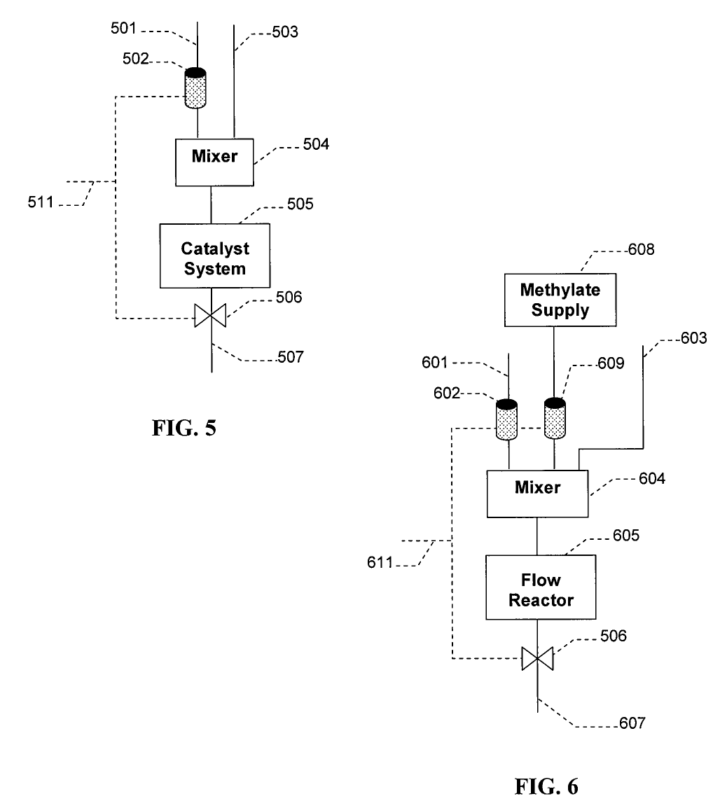

Please see FIG. 5 which illustrates the heterogeneous version of the finish reactor 115 from FIG. 1. The processed constituents entering the finish reactor via line 503 go to the mixing unit 504. The mixing unit combines that with methanol from the line 501, metered in proper proportion by the metering pump 502 and the computer control 100 connected via control line 511 connected to line 112. The mixed feedstock and methanol solution then pass to the heterogeneous catalyst system, 505. At this point the constituents entering via line 503 have a high methyl ester content sufficient to keep the constituents in a substantially single phase solution. This catalyst system at 505 is similar to the one in 204, FIG. 2.

Back pressure valve 506 is similar to back pressure valve 209. The output proceeds via line 507.

FIG. 6 illustrates the homogeneous version of the finish reactor 115 from FIG. 1. The processed feedstock entering the finish reactor via line 603 goes to the mixing unit 604. There methylate catalyst is mixed-in with that in a proportion controlled by the metering pump 609 from the supply 608. The methylate supply is premixed and dry to avoid introducing unwanted water.

Mixing unit 604 also combines methanol from line 601, connected to line 123, metered in proper proportion by the metering pump 602. The mixing unit also mixes in methyl ester sufficient to create a substantially single phase solution of all constituents, using the same proportion and conditions as used by mixing unit 202.

The mixed constituents in solution then pass to the flow reactor, 605, which provides static mixing in a particular temperature and pressure environment. Back pressure valve 606 is similar to back pressure valve 209. The output proceeds via line 607.

What is claimed is:

1. A method of increasing the throughput of esterification or transesterification reactions of multiphase immiscible biodiesel reaction constituents, comprising:
supplying the multiphase immiscible reaction constituents to a mixing unit;
providing a sufficient quantity of methyl ester at a sufficient temperature and pressure to form a substantially single phase solution in the mixing unit without the use of a co-solvent to be removed from the reaction constituents so as not to be present in a final biodiesel fuel;
passing the substantially single phase solution from the mixing unit to a reactor unit and maintaining the substantially single phase solution at a sufficient temperature and pressure to esterify or transesterify at least part of the reaction constituents.

2. The method of claim 1, wherein providing the methyl ester comprises gathering the methyl ester from an output of the reactor unit or an element coupled thereto and recirculating the methyl ester to an inlet of the mixing unit.

3. The method of claim 1 wherein the reaction constituents comprise methanol and triglyceride with at least 15% of the proportion of the total input to the mixing unit being methyl ester and wherein the mixing unit is maintained at a temperature of at least 60° C. and at least atmospheric pressure.

4. The method of claim 3 wherein the pressure and temperature are maintained as substantially the same throughout the reactor unit.

5. The method of claim 1 wherein the substantially single phase solution output from the mixing unit flows through a heterogeneous catalyst in the reactor unit.

6. The method of claim 1, further comprising:
supplying a catalyst consisting of dry sodium methylate in methanol as part of the reaction constituents to the mixing unit; and
passing the output from the reactor unit through a dry wash system comprising ion-resin beads, the ion resin beads converting a soap within the output to free fatty acid and adsorbing any remaining of the supplied catalyst.

7. The method of claim 6, further comprising:
supplying the triglyceride as a reaction constituent to the mixing unit; and
removing water from the triglyceride prior to supplying the triglyceride as a reaction constituent to the mixing unit.

8. The method of claim 7, further comprising removing water from the reaction constituents.

9. The method of claim 8, wherein removing water comprises distilling the reacted constituents after they have passed through at least one reactor unit.

10. The method of claim 1 wherein the reactor unit performs esterification or transesterification and wherein the method further comprises separating glycerine from the reacted constituents after they have passed through the reactor unit.

11. The method of claim 7, further comprising removing glycerin from the reacted constituents after they have passed through the reactor unit and wherein the methyl ester is gathered from the reacted constituents after they have passed through the reactor unit and the glycerin has been removed.

12. The method of claim 1, wherein a first mixing unit and reactor unit are provided and wherein high free fatty acid feedstock is processed therein, and wherein a second mixing unit and reactor unit are provided and low free fatty acid feedstock is processed therein.

13. A method of increasing the throughput of esterification or transesterification reactions of biodiesel fuel production, the method comprising:
separating and removing glycerin from the reaction product of a biodiesel feedstock and an alcohol;
gathering a portion of the reaction product after the glycerin has been separated and removed; and
causing a single phase solution to be formed of the biodiesel feedstock and the alcohol by:
recycling a quantity of the gathered portion of the reaction product containing a sufficient amount of methyl ester to form the single phase solution at a given temperature and pressure, to a mixing unit; and
maintaining the mixing unit at the given temperature and pressure thereby forming a substantially single phase solution of the biodiesel feedstock, alcohol, and recycled reaction product in the mixing unit.
